(19) United States
(12) Patent Application Publication
(10) Pub. No.: US 2010/0105833 A1
(43) Pub. Date: Apr. 29, 2010

(54) WATER-EMULSIFIABLE POLYISOCYANATES

(75) Inventors: Peter Keller, Spiesen-Elversberg (DE); Harald Schaefer, Mannheim (DE); Eva Wagner, Bad Duerkheim (DE); Thomas Zech, Bobenheim-Roxheim (DE); Angelika Maria Steinbrecher, Stuttgart (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/530,807

(22) PCT Filed: Mar. 14, 2008
(86) PCT No.: PCT/EP08/53088

§ 371 Date: Sep. 11, 2009

(30) Foreign Application Priority Data
Mar. 26, 2007 (EP) 07104873.0

(51) Int. Cl. C09D 175/04; C07C 263/00

(52) U.S. Cl. 524/839; 560/336

(57) ABSTRACT

The present invention describes a process for preparing water-emusifiable polyisocyanate.

The present invention describes a process for preparing water-emusifiable polyisocyanates.

EP 959087 A1 describes the preparation of water-emulsifiable polyisocyanates by reaction of polyisocyanates comprising at least two diisocyanate molecules with monofunctional polyethylene oxide polyether alcohols (paragraph [0017]). However, a separately prepared polyisocyanate is reacted with the component having an emulsifying action in each case (see paragraph [0042]).

A disadvantage of such a mode of operation is that such a preparation proceeds via two reaction steps and is therefore complicated.

EP 524500 A1 describes the preparation of polyisocyanates comprising isocyanurate groups and allophanate groups. To prepare polyisocyanates comprising allophanate groups, the reaction is carried out in the presence of monoalcohols which can be, among a number of possibilities, polyalkylene glycols. Various methods of preparing such polyisocyanates are described on page 5, lines 39 to 45: 1) a diisocyanate can firstly be reacted with the monoalcohol to form urethane groups and the actual polyisocyanate formation can be carried out subsequently or 2) the monoalcohol can be mixed with diisocyanate and subsequently reacted to form the polyisocyanate or 3) the monoalcohol can be added before or after, preferably after, the reaction of the diisocyanate to form the polyisocyanate or 4) the catalyst for preparing the polyisocyanate can be dissolved in monoalcohol or 5) polyisocyanate and monoallophanate can be mixed with one another.

The principle of the invention is described on page 5, lines 9-10 as being that urethane groups are introduced into a polyisocyanate by means of a monoalcohol and these are subsequently converted into allophanate groups. This corresponds to alternative 3) in which monoalcohol is added only after the reaction to form the polyisocyanate.

In all explicitly disclosed examples 1 to 7, on the other hand, diisocyanate is firstly reacted with the monoalcohol to form the urethane (preurethanization) and the reaction mixture obtained in this way is subsequently oligomerized (alternative 1).

The illustrative embodiments explicitly disclosed in examples 4, 5 and 7, in which the monoalcohols are polyethylene glycols, also follow alternative 1 in which the monoalcohol used is firstly reacted with the monomeric diisocyanate.

Both an evaluation and explicit disclosure are absent for the other alternatives.

DE-A 38 10 908 describes the formation of polyisocyanates which comprise isocyanurate groups and can optionally also be formed in the presence of alcohols (page 6, lines 38 to 48). However, in this case the alcohol only plays the role of a cocatalyst and is therefore used only in small amounts of from 0.05 to 1% by weight. The alcohols listed in DE-A 3810908 are not suitable for modifying the parent polyisocyanates so as to make them water-dispersible since they are not hydrophilic enough and/or are at least bifunctional. The monofunctional alcohols listed, viz. methanol, ethanol, butanol and phenol, are not sufficiently hydrophilic and the others have a higher functionality and thus lead to crosslinking of the polyisocyanate.

On the other hand, alcohols, in particular diols, are used in larger amounts as starting materials. Here, only a preurethanization process is disclosed (page 6, lines 42-48 and Example 3).

In contrast, water-emulsifiable polyisocyanates comprising monoalcohols having an emulsifying action are not disclosed at all.

DE 102004015983 A1 discloses a process for preparing polyisocyanates comprising allophanate groups. However, only alcohols having a mean functionality of>1.5 are disclosed as alcohols (paragraph [0013]) and only a preurethanization process in which urethane formation occurs separately from allophanate formation is disclosed (paragraphs [0024] and [0025]).

EP 56159 A describes the oligomerization of diisocyanates in the presence of basic alkali metal catalysts which are present as complex with a polyalkylene oxide. The latter can be, according to the explicitly disclosed examples, an unetherified, singly etherified or fully etherified alkoxylated diol or polyol. According to the teachings of EP 56159 A1, the amount of alcohols or complexing agents must be not more than 2 mol % based on the isocyanate groups used (page 12, line 23). This amount is not sufficient to produce water-dispersible polyisocyanates and EP 56159 A does not suggest any such preparation of water-dispersible polyisocyanates.

In contrast, it is stated on page 11, line 30 ff. that the amount of polyalkylene oxide and alcohol are deliberately selected so that the influence on the product is negligible. Only the use of polyalkylene oxide as complexing agents for basic alkali metal catalysts is disclosed.

In addition, the specification “not more than 2 mol %” is based on the sum of monohydric alcohols as solvents and polyalkylene oxides as complexing agents.

However, in the explicitly disclosed examples, significantly less monofunctional polyalkylene oxide is used per diisocyanate: Example 12: 0.023 mol %; Example 14: 0.019 mol %; Example 20: 0.12 mol %.

It was an object of the present invention to provide water-emulsifiable polyisocyanates which should display at least one of the following advantages: low viscosity, low color number, good dispersibility and high content of NCO groups at a dispersibility comparable to other water-emulsifiable polyisocyanates.

The object is achieved by water-emulsifiable polyisocyanates obtained by a process for preparing water-emulsifiable polyisocyanates comprising isocyanurate groups, in which

(A) at least one (cyclo)aliphatic diisocyanate,

(B) if appropriate at least one further diisocyanate other than (A) and

(C) at least one alkoxylated monoalcohol are simultaneously reacted with one another in the presence of at least one catalyst

(D) which is able to accelerate the formation of isocyanurate groups from isocyanate groups, the reaction is stopped on reaching the desired conversion and the unreacted diisocyanate (A) and if appropriate (B) are separated off from the reaction mixture, with the amount of alkoxylated monoalcohol (C) after the end of the reaction and removal of unreacted (A) and if appropriate (B) being at least 1.0 mol % based on the ratio of hydroxy groups to the sum of all NCO groups from the components (A) and (B).

The water-emulsifiable polyisocyanates obtained in this way have a good water-emulsifiability and a higher NCO content than comparably readily water-emulsifiable polyisocyanates. Furthermore, the water-emulsifiable polyisocyanates obtained according to the invention display a lower viscosity compared to products of this type for which, at comparable conversions, the reaction with an alkoxylated monoalcohol occurs only after isocyanurate formation (see Comparative example 4). Put in another way, the simultaneous reaction according to the invention enables higher conversions based on the formation of isocyanurate groups to be set in the production process in order to obtain products having the same viscosity than in the processes of the prior art. Moreover, the products obtained according to the invention display an improved dispersibility compared to products obtained by initial reaction with the alkoxylated monoalcohol (C) and only subsequent formation of the isocyanurates (Comparative example 2).

The component (A) is a (cyclo)aliphatic diisocyanate.

In the present text, the term “(cyclo)aliphatic” is used as an abbreviation for aliphatic or cycloaliphatic, preferably aliphatic.

Cycloaliphatic isocyanates are ones which comprise at least one cycloaliphatic ring system.

Aliphatic isocyanates are ones which comprise exclusively linear or branched chains, i.e. acyclic compounds.

The (cyclo)aliphatic diisocyanates (A) are preferably isocyanates having from 4 to 20 carbon atoms. Examples of usual aliphatic diisocyanates are tetramethylene diisocyanate, pentamethylene 1,5-diisocyanate, 2-methylpentane 1,5-diisocyanate, octamethylene diisocyanate, decamethylene diisocyanate, dodecamethylene diisocyanate, tetradecamethylene diisocyanate, derivatives of lysine diisocyanate, trimethylhexane diisocyanate and tetramethylhexane diisocyanate, and cycloaliphatic diisocyanates are, for example, 1,4-, 1,3- or 1,2-diisocyanatocyclohexane, isophorone diisocyanate, 4,4′- or 2,4′-di(isocyanatocyclohexyl)methane, 1,3- or 1,4-bis(isocyanato-methyl)cyclohexane, 2,4- or 2,6-diisocyanato-1-methylcyclohexane and 3 (or 4), 8 (or 9)-bis(isocyanatomethyl)tricyclo[5.2.1.0^{2.6}]decane isomer mixtures.

Diisocyanates (A) are particularly preferably at least one diisocyanate selected from the group consisting of hexamethylene 1,6-diisocyanate (HDI), isophorone diisocyanate (IPDI) 4,4′- and 2,4′-di(isocyanatocyclohexyl)methane.

The diisocyanate is very particularly preferably selected from the group consisting of hexamethylene 1,6-diisocyanate (HDI) and isophorone diisocyanate (IPDI), and is more particularly hexamethylene 1,6-diisocyanate.

It is possible to react one or more diisocyanates (A), for example from one to three diisocyanates (A), preferably one or two diisocyanates (A) and particularly preferably precisely one diisocyanate (A).

According to the invention, the process by which the diisocyanate (A) or (B) has been obtained and the purity in which it is used in the process of the invention play a minor role.

For the purposes of the present invention, it is possible to use both diisocyanates which are obtained by phosgenation of the corresponding amines and ones which are prepared without use of phosgene, i.e. by phosgene-free processes. Such phosgene-free processes are described, for example, in EP-A-0 126 299 (U.S. Pat. No. 4,596,678), EP-A-126 300 (U.S. Pat. No. 4,596,679) and EP-A-355 443 (U.S. Pat. No. 5,087,739). The diisocyanates are prepared by reacting the (cyclo)aliphatic diamines with, for example, urea and alcohols to form (cyclo)aliphatic biscarbamic esters and thermally dissociating these into the corresponding diisocyanates and alcohols. The synthesis is usually carried out continuously in a circulatory process and if appropriate in the presence of N-unsubstituted carbamic esters, dialkyl carbonates and other by-products recirculated from the reaction process. Diisocyanates obtained in this way generally have a very low or even unmeasurable proportion of chlorinated compounds, which is desirable in, for example, electronics applications.

In an embodiment of the present invention, the diisocyanates (A) and if appropriate (B) have a total content of hydrolyzable chloride of less than 200 ppm, preferably less than 120 ppm, particularly preferably less than 80 ppm, very particularly preferably less than 50 ppm, in particular less than 15 ppm and especially less than 10 ppm. This can, for example, be measured by the ASTM method D4663-98. However, it is of course also possible to use diisocyanates (A) and if appropriate (B) having a higher chlorine content, for example up to 500 ppm.

It goes without saying that mixtures of diisocyanates which have been obtained by reaction of (cyclo)aliphatic diamines with, for example, urea and alcohols and dissociation of the resulting (cyclo)aliphatic biscarbamic esters with diisocyanates obtained by phosgenation of the corresponding amines can also be used.

According to the invention, the content of isomeric compounds in the diisocyanate (A) does not play a critical role.

Thus, hexamethylene 1,6-diisocyanate can comprise, for example, a small proportion of 2- and/or 3-methylpentamethylene 1,5-diisocyanate.

1-lsocyanato-3,3,5-trimethyl-5-(isocyanatomethyl)cyclohexane (isophorone diisocyanate, IPDI) is usually present as a mixture of cis and trans isomers, generally in a ratio of from about 60:40 to 80:20 (w/w), preferably in a ratio of from about 70:30 to 75:25 and particularly preferably in a ratio of about 75:25.

Dicyclohexylmethane 4,4′-diisocyanate can likewise be present as a mixture of the various cis and trans isomers.

The component (B) can be at least one isocyanate other than (A), for example aromatic or araliphatic isocyanates (B), preferably aromatic isocyanates (B).

Aromatic isocyanates are ones which comprise at least one aromatic ring system.

Araliphatic isocyanates are ones which comprise an aromatic ring with isocyanate groups bound thereto via aliphatic chains.

The isocyanates (B) are preferably diisocyanates which bear precisely two isocyanate groups. However, they can in principle also be monoisocyanates having precisely one isocyanate group.

Higher isocyanates having an average of more than 2 isocyanate groups are in principle also possible. Examples of suitable isocyanates of this type are triisocyanates such as triisocyanatononane, 2,4,6-triisocyanatotoluene, triphenylmethane triisocyanate and 2,4,-diisocyanatophenyl 4′-isocyanatodiphenyl ether and the mixtures of diisocyanates, triisocyanates and higher polyisocyanates which are obtained, for example, by phosgenation of appropriate aniline-formaldehyde condensates and represent polyphenyl polyisocyanates having methylene bridges.

The diisocyanates (B) are preferably isocyanates having from 4 to 20 carbon atoms. Examples of aromatic diisocyanates are tolylene 2,4- or 2,6-diisocyanate and their isomer mixtures, m- or p-xylylene diisocyanate, 2,4′- or 4,4′-diisocyanatodiphenylmethane and their isomer mixtures, phenylene 1,3- or 1,4-diisocyanate, 1-chlorophenylene 2,4-diisocyanate, naphthylene 1,5-diisocyanate, biphenylene 4,4′-diisocyanate, 4,4′-diisocyanato-3,3′-dimethylbiphenyl, 3-methyldiphenylmethane 4,4′-diisocyanate, tetramethylxylylene diisocyanate, 1,4-diisocyanatobenzene or diphenyl ether 4,4′-diisocyanate.

Particular preference as diisocyanate (B) is given to tolylene 2,4- or 2,6-diisocyanate and their isomer mixtures and tetramethyl-xylylene diisocyanate, very particularly preferably tolylene 2,4- or 2,6-diisocyanate and their isomer mixtures.

Mixtures of the isocyanates mentioned can also be present.

The component (C) comprises monofunctional polyalkylene oxide polyether alcohols which are reaction products of suitable starter molecules with polyalkylene oxides.

Suitable starter molecules for preparing monohydric polyalkylene oxide polyether alcohols are thiol compounds, monohydroxy compounds of the general formula

R¹—O—H

or secondary monoamines of the general formula

R²R³N—H,

where
R¹, R² and R³ are each, independently of one another, C₁-C₂₀-alkyl, C₂-C₂₀-alkyl which may optionally be interrupted by one or more oxygen and/or sulfur atoms and/or one or more substituted or unsubstituted imino groups, C₆-C₁₂-aryl, C₅-C₁₂-cycloalkyl or a five- or six-membered, oxygen-, nitrogen- and/or sulfur-comprising heterocycle or R² and R³ together form an unsaturated, saturated or aromatic ring which may optionally be interrupted by one or more oxygen and/or sulfur atoms and/or one or more substituted or unsubstituted imino groups, where the radicals mentioned may also be substituted by functional groups, aryl, alkyl, aryloxy, alkyloxy, halogen, heteroatoms and/or heterocycles.

Preference is given to R¹, R² and R³ each being, independently of one another, C₁-C4-alkyl, i.e. methyl, ethyl, isopropyl, n-propyl, n-butyl, isobutyl, sec-butyl or tert-butyl, with particular preference being given to R¹, R² and R³ each being methyl.

Examples of suitable monofunctional starter molecules are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, cyclopentanol, the isomeric methylcyclohexanols or hydroxymethylcyclohexane, 3-ethyl-3-hydroxy-methyloxetane or tetrahydrofurfuryl alcohol; unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol; secondary monoamines such as dimethylamine, diethylamine, dipropylamine, diisopropylamine, di-n-butylamine, diisobutylamine, bis(2-ethylhexyl)amine, N-methylcyclohexylamine and N-ethylcyclohexylamine or dicyclohexylamine, heterocyclic secondary amines such as morpholine, pyrrolidine, piperidine or 1H-pyrazole, and also amino alcohols such as 2-dimethylaminoethanol, 2-diethylaminoethanol, 2-diisopropylaminoethanol, 2-dibutylaminoethanol, 3-(dimethylamino)-1-propanol or 1-(dimethylamino)-2-propanol.

Examples of amine-initiated polyethers are the Jeffamine® M series, which are methyl-capped polyalkylene oxides having an amino function, e.g. M-600 (XTJ-505) having a propylene oxide (PO)/ethylene oxide (EO) ratio of about 9:1 and a molar mass of about 600, M-1000 (XTJ-506): PO/EO ratio 3:19, molar mass about 1000, M-2005 (XTJ-507): PO/EO ratio 29:6, molar mass about 2000 or M-2070: PO/EO ratio 10:31, molar mass about 2000.

Alkylene oxides suitable for the alkoxylation reaction are ethylene oxide, propylene oxide, isobutylene oxide, vinyloxirane and/or styrene oxide, which can be used in any order or in admixture in the alkoxylation reaction.

Preferred alkylene oxides are ethylene oxide, propylene oxide and mixtures thereof, with particular preference being given to ethylene oxide.

Preferred polyether alcohols are those based on polyalkylene oxide polyether alcohols which have been prepared using saturated aliphatic or cycloaliphatic alcohols of the abovementioned type as starter molecules. Very particular preference is given to those based on polyalkylene oxide polyether alcohols which have been prepared using saturated aliphatic alcohols having from 1 to 4 carbon atoms in the alkyl radical. Particular preference is given to methanol-initiated polyalkylene oxide polyether alcohols.

The monohydric polyalkylene oxide polyether alcohols have an average of generally at least 2 alkylene oxide units, preferably at least 5 alkylene oxide units, per molecule, particularly preferably at least 7 alkylene oxide units and very particularly preferably at least 10 alkylene oxide units, in particular ethylene oxide units.

The monohydric polyalkylene oxide polyether alcohols have an average of generally up to 50 alkylene oxide units per molecule, preferably up to 45 alkylene oxide units, particularly preferably up to 40 alkylene oxide units and very particularly preferably up to 30 alkylene oxide units, in particular ethylene oxide units.

The molecular weight of the monohydric polyalkylene oxide polyether alcohols is preferably up to 4000 g/mol, particularly preferably not more than 2000 g/mol, very particularly preferably not less than 250 g/mol and in particular 500±100 g/mol.

Preferred polyether alcohols are thus compounds of the formula

R¹—O—[Xᵢ—]ₖ—H

where

R¹ is as defined above,

k is an integer from 5 to 40, preferably from 7 to 20 and particularly preferably from 10 to 15, and

each Xᵢ for i=1 to k can be selected independently from the group consisting of —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂—CHVin—O—, —CHVin—CH₂—O—, —CH₂—CHPh—O— and —CHPh—CH₂—O— preferably from the group consisting of —CH₂—CH₂—O—, —CH₂—CH(CH₃)—O— and —CH(CH₃)—CH₂—O— and particularly preferably —CH₂—CH₂—O—, where Ph is phenyl and Vin is vinyl.

The polyalkylene oxide polyether alcohols are generally prepared by alkoxylation of the starter compounds in the presence of a catalyst, for example an alkali metal or alkaline earth metal hydroxide, oxide, carbonate or hydrogencarbonate.

The preparation of the polyalkylene oxide polyether alkyls can also be carried out with the aid of multimetal cyanide compounds, frequently also referred to as DMC catalysts, which have been known for a long time and are widely described in the literature, for example in U.S. Pat. No. 3,278,457 and in U.S. Pat. No. 5,783,513.

The DMC catalysts are usually prepared by reacting a metal salt with a cyanometalate compound. To improve the properties of the DMC catalysts, it is customary to add organic ligands during and/or after the reaction. A description of the preparation of DMC catalysts may be found, for example, in U.S. Pat. No. 3,278,457.

Typical DMC catalysts have the following general formula:

M¹ₐ[M₂(CN)_{b}]_{d}·fM¹ⱼXₖ·h(H₂O) eL·zP

where

M¹ is a metal ion selected from the group consisting of Zn²⁺, Fe²⁺, Fe³⁺, Co²⁺, Co³⁺, Ni²⁺, Mn²⁺, Sn²⁺, Sn⁴⁺, Pb²⁺, Al³⁺, Sr²⁺, Cr³⁺, Cd²⁺, Cu²⁺, La³⁺, Ce³⁺, Ce⁴⁺, Eu³⁺, Mg²⁺, Ti⁴⁺, Ag⁺, Rh²⁺, Ru²⁺, Ru³⁺, Pd²⁺,

M² is a metal ion selected from the group consisting of Fe²⁺, Fe³⁺, Co²⁺, Co³⁺, Mn²⁺, Mn³⁺, Ni²⁺, Cr²⁺, Cr³⁺, Rh³⁺, Ru²⁺, Ir³⁺

and M¹ and M² are identical or different,

X is an anion selected from the group consisting of halide, hydroxide, sulfate, hydrogensulfate, carbonate, hydrogencarbonate, cyanide, thiocyanate, isocyanate, cyanate, carboxylate, oxalate, nitrate and nitrite (NO₂—) or a mixture of two or more of the abovementioned anions or a mixture of one or more of the abovementioned anions with an uncharged species selected from among CO, H₂O and NO,

Y is an anion which is different from X and is selected from the group consisting of halide, sulfate, hydrogensulfate, disulfate, sulfite, sulfonate (=RSO₃— where R=C1-C20-alkyl , aryl, C1-C20-alkylaryl), carbonate, hydrogencarbonate, cyanide, thiocyanate, isocyanate, isothiocyanate, cyanate, carboxylate, oxalate, nitrate, nitrite, phosphate, hydrogenphosphate, dihydrogenphosphate, diphosphate, borate, tetraborate, perchlorate, tetrafluoroborate, hexafluorophosphate, tetraphenylborate,

L is a water-miscible ligand selected from the group consisting of alcohols, aldehydes, ketones, ethers, polyethers, esters, polyesters, polycarbonate, ureas, amides, nitriles and sulfides or a mixture thereof,

P is an organic additive selected from the group consisting of polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyalkyl acrylates, polyalkyl methacrylates, polyvinyl methyl ether, polyvinyl ethyl ether, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkylenimines, maleic acid and maleic anhydride copolymers, hydroxyethylcellulose, polyacetates, ionic surface-active and interface-active compounds, bile acids or their salts, esters or amides, carboxylic esters of polyhydric alcohols and glycosides

and

a, b, d, g, n, r, s, j, k and t are integers or fractions greater than zero, e, f, h and z are integers or fractions greater than or equal to zero,

where

a, b, d, g, n, j, k and r and also s and t are selected so that the compound is electrically neutral,

M³ is hydrogen or an alkali metal or alkaline earth metal and

M⁴ is an alkali metal ion or an ammonium ion (NH₄₊) or alkylammonium ion (R₄N⁺, R₃NH⁺, R₂NH₂⁺, RNH₃⁺ where R=C1-C20-alkyl).

In a particularly preferred embodiment of the invention, M¹ is Zn²⁺ and M² is Co³⁺ or Co²⁺.

The metals M¹ and M² are, in particular, identical when they are cobalt, manganese or iron.

The residues of the catalyst can remain in the product obtained or can be neutralized by means of an acid, preferably hydrochloric acid, sulfuric acid or acetic acid, with the salts subsequently being able to be removed, preferably by, for example, washing or by means of ion exchanger. If appropriate, a partial neutralization can be carried out and the product can be used further without further removal of the salts.

The catalyst (D) is, according to the invention, a compound which is able to accelerate the formation of isocyanurate groups from isocyanate groups. Apart from isocyanurate groups, further polyisocyanate units can be formed, for example uretdione, biuret, urethane, allophanate, oxadiazinetrione, iminooxadiazinetrione, uretonimine or carbodiimide groups. The catalyst preferably catalyzes the formation of urethane groups in particular, but this is not absolutely necessary for the process of the invention.

Trimerization catalysts which are suitable for the process of the invention include, for example,

aziridine derivatives in combination with tertiary amines of the type described in U.S. Pat. No. 3,919,218;

quaternary ammonium carboxylates of the type described in the U.S. Pat. Nos. 4,454,317 and 4,801,663;

quaternary ammonium phenoxides having a zwitterionic structure of the type described in U.S. Pat. No. 4,335,219;

ammonium phosphonates and phosphates of the type described in U.S. Pat. No. 4,499,253;

alkali metal phenoxides of the type described in GB-B 1,391,066 or GB-B 1,386,399;

alkali metal carboxylates, for example cobalt naphthenate, sodium benzoate, sodium acetate, potassium formate and as described in DE-A 3,219,608;

basic alkali metal salts complexed by acyclic organic compounds, as are described in U.S. Pat. No. 4,379,905, for instance potassium acetate complexed by a polyethylene glycol comprising an average of from 5 to 12 ethylene oxide units;

basic alkali metal salts complexed with crown ethers, as are described in U.S. Pat. No. 4,487,928;

compounds comprising aminosilyl groups, e.g. aminosilanes, diaminosilanes, silylureas and silazanes, as are described in U.S. Pat. No. 4,412,073;

mixtures of alkali metal fluorides and quaternary ammonium or phosphonium salts, as are described in EP-A 355479,

tertiary amines, for example triethylamine, N,N-dimethylbenzylamine, triethylenediamine, 2,4,6-tris(dimethylaminomethyl)phenol and 1,3,5-tris(dimethyl-aminopropyl)-S-hexahydrotriazine,

N-heterocyclic carbenes (NHCs) as in WO 2005/113626,

alkali metal oxides, alkali metal hydroxides and strong organic bases, e.g. alkali metal alkoxides,

tin, zinc and lead salts of alkylcarboxylic acids,

organic salts of the formula (A)ₙ—R—O—CO—⊖M⊕ as described in U.S. Pat. No. 3,817,939, where:

A is a hydroxyl group or a hydrogen atom,

n is from 1 to 3,

R is a polyfunctional linear or branched, aliphatic or aromatic hydrocarbon radical and M⊕ is a cation, e.g. an alkali metal cation or a quaternary ammonium cation, e.g. tetraalkylammonium, and

quaternary hydroxyalkylammonium compounds of the formula

R⁴,R⁵,R⁶N⊕—CH₂—CH(OH)—R^{7 ⊖O—(CO)—R}⁸

as catalyst as described in DE-A-26 31 733 (U.S. Pat. No. 4,040,992).

Particularly useful catalysts for the process are quaternary ammonium salts corresponding to the formula

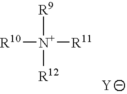

where

Y⊖=carboxylate (R¹³COO⁻), fluoride (F⁻), carbonate (R¹³O(CO)O⁻) or hydroxide (OH⁻), as are described for Y⁻=OH⁻ in U.S. Pat. No. 4,324,879 and in DE-A-2,806,731 and 2,901,479.

The radical Y⊖ is preferably a carboxylate, carbonate or hydroxide and particularly preferably a carboxylate or hydroxide.

In these, R¹³ is hydrogen, C₁-C₂₀-alkyl, C₆-C₁₂-aryl or C₇-C₂₀-arylalkyl, which may in each case optionally be substituted.

R¹³ is preferably hydrogen or C₁-C₈-alkyl.

Among the abovementioned catalysts, those comprising metal ions, for example alkali metal or alkaline earth metal ions, in particular alkali metal ions, are less preferred for the process of the invention.

Among the catalysts listed, preferred catalysts for the process of the invention are those which have quaternary ammonium ions as cations, particularly preferably quaternary ammonium ions which bear a βhydroxyalkyl unit.

Preferred quaternary ammonium salts are those in which the radicals R⁹ to R¹² are identical or different alkyl groups having from 1 to 20, preferably from 1 to 4, carbon atoms, which may optionally be substituted by hydroxyl or phenyl groups.

It is also possible for two of the radicals R⁹ to R¹² together with the nitrogen atom and, if appropriate, a further nitrogen or oxygen atom to form a heterocyclic, five-, six- or seven-membered ring. The radicals R⁹ to R¹¹ can in this case also be ethylene radicals which together with the quaternary nitrogen atom and a further tertiary nitrogen atom form a bicyclic triethylenediamine structure, provided that the radical R¹² is then a hydroxyalkyl group which has from 2 to 4 carbon atoms and in which the hydroxyl group is preferably located in the 2 position relative to the quaternary nitrogen atom. The hydroxy-substituted radical or radicals can also comprise other substituents, for example C₁-C₄-alkyloxy substituents.

The ammonium ions can also be part of a monocyclic or polycyclic ring system, for example be derived from piperazine, morpholine, piperidine, pyrrolidine, quinuclidine or diazabicyclo[2.2.2]octane.

Examples of groups R⁹ to R¹² having from 1 to 20 carbon atoms are, independently of one another, methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hetadecyl, octadecyl, 1,1-dimethylpropyl, 1,1-dimethylbutyl, 1,1,3,3-tetramethylbutyl, benzyl, 1-phenylethyl, 2-phenylethyl, a,a-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, chloromethyl, 2-chloroethyl, trichloromethyl, trifluoromethyl, 1,1-dimethyl-2-chloroethyl, 2-methoxyisopropyl, 2-ethoxyethyl, 2-hydroxyethyl, 2-hydroxypropyl, 3-hydroxypropyl, 4-hydroxybutyl, 6-hydroxyhexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, methylnaphthyl, isopropylnaphthyl, chloronaphthyl, 2,6-dimethylphenyl, 2,4,6-trimethylphenyl, 2,6-dimethoxyphenyl, 2,6-dichlorophenyl, cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, chlorocyclohexyl, dichlorocyclohexyl, dichlorocyclopentyl, norbornyl or norbornenyl.

Preference is given to the radicals R⁹ to R¹² each being, independently of one another, C₁-C₄-alkyl. R¹² can additionally be benzyl or a radical of the formula

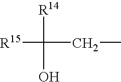

where R¹⁴ and R¹⁵ can each be, independently of one another, hydrogen or C₁-C₄-alkyl.

Particularly preferred radicals R⁹ to R¹² are, independently of one another, methyl, ethyl and n-butyl and in the case of R¹² additionally benzyl, 2-hydroxyethyl and 2-hydroxypropyl.

The following catalysts can preferably be used for the process of the invention:

quaternary ammonium hydroxides, preferably N,N,N-trimethyl-N-benzylammonium hydroxide and N,N,N-trimethyl-N-(2-hydroxypropyl)ammonium hydroxide, as described in DE-A-38 06 276.

Hydroxyalkyl-substituted quaternary ammonium hydroxides as described in EP-A-10 589 (U.S. Pat. No. 4,324,879).

Organic metal salts of the formula (A)ₙ—R—O—CO—O⊖M⊕ as described in U.S. Pat. No. 3,817,939, where A is a hydroxyl group or a hydrogen atom, n is from 1 to 3, R is a polyfunctional linear or branched, aliphatic or aromatic hydrocarbon radical and M is a cation of a strong base, e.g. an alkali metal cation or a quaternary ammonium cation, e.g. tetraalkylammonium,

In the present text,

C₁-C₂₀-alkyl which may optionally be substituted by aryl, alkyl, aryloxy, alkyloxy, heteroatoms and/or heterocycles is, for example, methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hetadecyl, octadecyl, eicosyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl,

C₆-C₁₂-aryl which may optionally be substituted by aryl, alkyl, aryloxy, alkyloxy, heteroatoms and/or heterocycles is, for example, phenyl, tolyl, xylyl, a-naphthyl, 13-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl,

C₅-C₁₂-cycloalkyl which may optionally be substituted by aryl, alkyl, aryloxy, alkyloxy, heteroatoms and/or heterocycles is, for example, cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl,

divalent C₂-C₉-alkylene radicals, which may also be a constituent of an arylene or cycloalkylene radical, are, for example, 1,2-ethylene 1,2-propylene, 1,3-propylene, 1,6-hexylene, 2,2,4-trimethylhexylene, 1,4-cyclohexylene, isopropylidene-1,4-dicyclohexylene, 1,2-, 1,3- or 1,4-phenylene, 4,4′-biphenylene, 4,4′-bisphenylmethylene, 1,3-, 1,4- or 1,5-naphthylene, 3,3′-dimethyl-4,4′-diphenylene, 3,3′-dichloro-4,4′-diphenylene, 2,4- or 2,6-pyridyl, 1,4-anthraquinonediyl, m- or p-tolylene, 4,6-dimethyl-1,3-phenylene, 4,6-dichloro-1,3-phenylene, 5-chloro-1,3-phenylene, 5-hydroxy-1,3-phenylene, 5-methoxy-1,3-phenylene, 2,3-dimethyl-1,4-phenylene, m- or p-xylylene, methylenedi-p-phenylene, isopropylidenedi-p-phenylene, thiodi-p-phenylene, dithiodi-p-phenylene, sulfodi-p-phenylene, carbonyldi-p-phenylene, and

C₁-C₄-alkyl is, for example, methyl, ethyl, propyl, isopropyl, n-butyl, sec-butyl or tert-butyl.

These quaternary ammonium catalysts are prepared in a known manner by reacting a tertiary amine with an alkylene oxide in an aqueous-alcoholic medium (cf. U.S. Pat. No. 3,995,997, column 2, lines 19-44).

Examples of suitable tertiary amines are trimethylamine, tributylamine, 2-dimethylaminoethanol, triethanolamine, dodecyldimethylamine, N,N-dimethylcyclohexylamine, N-methylpyrrolidine, N-methylmorpholine and 1,4-diazabicyclo[2.2.2]octane. Examples of suitable alkylene oxides are ethylene oxide, propylene oxide, 1,2-butylene oxide, styrene oxide and methoxypropylene, ethoxypropylene or phenoxypropylene oxide.

The most preferred catalysts (D) are N,N,N,N-tetramethylammonium hydroxide, N,N,N,N-tetraethylammonium hydroxide, N,N,N,N-tetra-n-butylammonium hydroxide, N,N,N-trimethyl-N-(2-hydroxyethyl)ammonium hydroxide, N,N,N-trimethyl-N-(2-hydroxypropyl)ammonium hydroxide, N,N,N-trimethyl-N-benzylammonium hydroxide, N-(2-hydroxypropyl)-N,N,N-trimethylammonium 2-ethylhexanoate (DABCO TMR®) and N-(2-hydroxypropyl)-N,N,N-trimethylammonium 2-formate (DABCO TMR®-2) from Air Products.

Preference is also given to trimerization catalysts as are known from DE 10 2004 012571 A1, there in particular paragraphs [0017] to [0027], and from EP-A1 668 271, there in particular from page 4, line 16 to page 6, line 47, which are hereby incorporated by reference into the present disclosure.

The catalysts (D) are generally used in amounts of from about 0.0005 to 5% by weight, preferably from about 0.001 to 2% by weight, based on the isocyanate used. If, for example, a preferred catalyst such as N,N,N-trimethyl-N-(2-hydroxypropyl)ammonium hydroxide is used, amounts of from about 0.0005 to 1% by weight, preferably from about 0.001 to 0.02% by weight, based on the starting isocyanate, are generally sufficient.

The catalysts can be used in pure form or in solution. To improve handling, the catalyst can be dissolved in a solvent. Solvents suitable for this purpose are, for example, alcohols, in particular diols, ketones, ethers and esters. The solvents listed in this text which are inert toward isocyanate groups are suitable as solvents, depending on the type of catalyst. Dimethylformamide or dimethyl sulfoxide can likewise be used as solvent for the catalysts.

For example, the abovementioned catalysts DABCO TMR® and DABCO TMR®-2 are preferably used as an about 33-75% strength by weight solution in diethylene glycol, dipropylene glycol or preferably ethylene glycol.

The use of monoalcohols, preferably alkanols such as methanol, ethanol, isopropanol, n-propanol, n-butanol, isobutanol, sec-butanol, tert-butanol, n-hexanol, n-heptanol, n-octanol, n-decanol, n-dodecanol (lauryl alcohol) or 2-ethylhexanol, in amounts which do not significantly influence the properties of the product mixture is also conceivable.

Further suitable catalysts are aminosilyl compounds as are described in U.S. Pat. No. 4,412,073, there in particular from column 3, lines 2 to 33 and column 4, line 1 to column 6, line 4, which is hereby expressly incorporated by reference into the present disclosure. Among these aminosilyl compounds, methylaminotrimethylsilane, dimethylaminotrimethylsilane, diethylaminotrimethylsilane, dibutylaminotrimethylsilane, diethylaminodimethylvinylsilane, diethylaminodimethylphenylsilane, bis(dimethylamino)dimethylsilane, bis(diethylamino)dimethylsilane, bis(dibutylamino)dimethylsilane, bis(dimethylamino)methylphenylsilane, N-methyl-N-trimethylsilyl-N′-methyl-N′-butylurea, N-methyl-N-trimethylsilyl-N′,N′-dimethylurea, N-ethyl-N-trimethylsilyl-N′,N′-dimethylurea and N-butyl-N-trimethylsilyl-N′,N′-dimethylurea are particularly useful.

Very particularly useful silyl compounds are hexamethyldisilazane, heptamethyldisilazane, hexaethyldisilazane, 1,3-diethyl-1,1,3,3-tetramethyldisilazane, 1,3-divinyl-1,1,3,3-tetramethyldisilazane, 1,3-diphenyl-1,1,3,3-tetramethyldisilazane and in particular hexamethyldisilazane.

Furthermore, ionic liquids, for example those having a choline or triazolate structure as described, for example, in WO 2006/084880, are suitable as catalysts.

Further suitable catalysts are hydrogen (poly)fluoride, e.g. compounds having the composition [M(nF·(HF)ₘ)], where M is an n-fold charged cation or an n-valent radical, e.g. tetraorganylammonium or tetraorganylphosphonium hydrogen polyfluoride as described, for example, in EP 896009 or DE 3902078, if appropriate diluted in monohydric or polyhydric alcohols or aprotic solvents. Likewise suitable are salts of polyfluorinated carboxylates having preferably quaternary ammonium or phosphonium cations as counterion as described in EP 1645577, if appropriate diluted in monohydric or polyhydric alcohols or aprotic solvents. These catalysts lead to unsymmetrical, low-viscosity isocyanurate structures which in combination with the low-viscosity products according to the invention described here have a particularly low viscosity.

It can also be conceivable to use if appropriate small amounts of monoalcohols (E) which are different from (C) and if appropriate small amounts of diols or polyols (F) as formative components of the water-emulsifiable polyisocyanates.

Monoalcohols (E) are, for example, C₁-C₂₀-alkanols, C₅-C₁₂-cycloalkanols and C₁-C₄-alkyloxy-C₁-C₂₀-alkanols; among these preferably C₁-C₄-alkyloxy-C₁-C₄-alkanols.

Examples are methanol, ethanol, isopropanol, n-propanol, n-butanol, isobutanol, sec-butanol, tert-butanol, n-hexanol, n-heptanol, n-octanol, n-decanol, n-dodecanol (lauryl alcohol), 2-ethylhexanol, n-pentanol, stearyl alcohol, cetyl alcohol, lauryl alcohol, ethylene glycol monomethyl ether, ethylene glycol monoethyl ether, 1,3-propanediol monomethyl ether, cyclopentanol, cyclohexanol, cyclooctanol, cyclododecanol.

Diols and polyols (F) are alcohols having a functionality of 2 or above, preferably from 2 to 6, particularly preferably from 2 to 4, very particularly preferably from 2 to 3 and in particular precisely 2.

Examples are trimethylolpropane, neopentyl glycol, pentaerythritol, 1,4-butanediol, 1,6-hexanediol, 1,2-propanediol, 1,3-propanediol, 2-ethyl-1,3-propanediol, 2-methyl-1,3-propanediol, ethylene glycol, diethylene glycol, triethylene glycol, tetraethylene glycol, pentaethylene glycol, glycerol, 1,2-dihydroxypropane, 2,2-dimethyl-1,2-ethanediol, 1,2-butanediol, 1,3-butanediol, 1,4-butanediol, 2,4-pentanediol, 3-methyl-1,5-pentanediol, 2,2,4-trimethyl-1,5-pentanediol, 2-methyl-2,4-pentanediol, 2-methyl-1,3-pentanediol, 2-ethyl-1,3-hexanediol, 2-propyl-1,3-heptanediol, 2,4-diethyl-1,3-octanediol, the neopentyl glycol ester of hydroxypivalic acid, ditrimethylolpropane, dipentaerythritol, 2,2-bis(4-hydroxycyclohexyl)propane, 1,1-, 1,2-, 1,3- and 1,4-cyclohexanedimethanol, 1,2-, 1,3- or 1,4-cyclohexanediol or mixtures thereof.

The use of polyTHF having a molecular weight in the range from 162 to 4500, poly-1,3-propanediol having a molecular weight in the range from 134 to 400 or polyethylene glycol having a molecular weight in the range from 238 to 458 is also conceivable.

The water-emulsifiable polyisocyanates after separation of monomeric isocyanate (A) used and, if appropriate, (B) are generally made up of the abovementioned formative components as follows:

(A) from 25 to 100 mol %, preferably from 34 to 100 mol %, particularly preferably from 66 to 100 mol %, very particularly preferably from 75 to 100 mol %, in particular from 90 to 100 mol %, especially from 95 to 100 mol % and even 100 mol % (based on the sum of all NCO groups in the water-emulsifiable polyisocyanate formed),

(B) from 0 to 75 mol %, preferably from 0 to 66 mol %, particularly preferably from 0 to 34 mol %, very particularly preferably from 0 to 25 mol %, in particular from 0 to 10 mol %, especially from 0 to 5 mol % and even 0 mol % (based on the sum of all NCO groups in the water-emulsifiable polyisocyanate),

(C) at least 1.0 mol %, preferably at least 1.5 mol %, particularly preferably at least 2.0 mol %, very particularly preferably at least 2.3 mol %, in particular at least 2.5 mol %, especially at least 3 mol %, often at least 4 mol % and even at least 5 mol %, and up to 25 mol %, preferably up to 20 mol %, particularly preferably up to 15 mol % and very particularly preferably up to 10 mol % (based on the ratio of hydroxy groups to the sum of all NCO groups from the components (A) and (B)),

(E) from 0 to 10 mol %, preferably from 0 to 8 mol %, particularly preferably from 0 to 5 mol % and very particularly preferably 0 mol % (based on the ratio of hydroxy groups to the sum of all NCO groups from the components (A) and (B)) and

(F) from 0 to 10 mol %, preferably from 0 to 8 mol %, particularly preferably from 0 to 5 mol % and very particularly preferably 0 mol % (based on the ratio of hydroxy groups to the sum of all NCO groups from the components (A) and (B)).

The NCO functionality of the water-emulsifiable polyisocyanate is generally at least 1.8 and can be up to 8, preferably from 1.8 to 5 and particularly preferably from 2 to 4.

The content of isocyanate groups after the oligomerization and removal of monomers, calculated as NCO=42 g/mol, is generally from 5 to 25% by weight.

According to the invention, it is critical that the reaction of the component (C) with the components (A) and, if appropriate, (B) and the formation of isocyanurate groups take place simultaneously with one another. For the purposes of the present text, this means that not more than 30 mol %, preferably not more than 25 mol %, particularly preferably not more than 20 mol % and very particularly preferably not more than 15 mol %, of the hydroxy groups in (C) have reacted to form urethane and, if appropriate, allophanate groups before commencement of the formation of isocyanurate groups from (A) and, if appropriate, (B) and at the same time not more than 15 mol %, preferably not more than 12 mol %, particularly preferably not more than 10 mol %, very particularly preferably not more than 8 mol % and in particular not more than 5 mol %, of the isocyanate groups comprised in (A) and, if appropriate, (B) have reacted to form isocyanurate groups before commencement of the formation of urethane groups.

If (E) and/or (F) are comprised as further formative components, it is of only minor importance for the purposes of the invention whether these components react before, during or after isocyanurate formation.

The reaction of the formative components with one another can be carried out either batchwise or continuously and, for example, as follows:

The components (A) and, if appropriate, (B) comprising isocyanate groups are placed in a reaction vessel and the catalyst (D) and the hydroxyl-comprising components (C) and, if appropriate, (E) and/or (F) are introduced simultaneously or without a large difference in the time at which they are added. In a preferred embodiment, at least one hydroxyl-comprising component can be used as solvent for the catalyst (D).

As an alternative, the components comprising isocyanate groups and the hydroxyl-comprising components can be mixed with one another under conditions under which the formation of urethane groups does not yet occur to a significant extent. The catalyst (D) is then added to this mixture either in portions or all at once.

The reaction according to the invention then occurs while stirring at a temperature in the range from 40° C. to 170° C., preferably from 45° C. to 160° C., particularly preferably from 50 to 150° C. and very particularly preferably from 60 to 140° C.

Heating can be, for example, via jacket heating, welded-on full tubes or half tubes, via internal tubes or plates and/or via a circuit having an external heat exchanger, e.g. shell-and-tube or plate heat exchanger. According to the invention, preference is given to using a circuit having an external heat exchanger. Uniform mixing of the reaction solution is effected in a known way, e.g. by stirring, pump circulation, forced convection, natural convection, preferably by forced convection or natural convection.

The mean reaction time is generally at least 2 minutes, preferably at least 5 minutes, particularly preferably at least 10 minutes, very particularly preferably at least 15 minutes and in particular at least 20 minutes, and can be up to 7 hours, preferably up to 90 minutes, particularly preferably up to 60 minutes, very particularly preferably up to 45 minutes and in particular up to 30 minutes.

The reaction zone can be backmixed or not backmixed; combinations thereof are also conceivable.

The reaction zones can be, for example, a plurality of stirred vessels connected in series (cascade of stirred vessels) through to flow characteristics of a tube reactor, or a tube reactor or at least one stirred vessel which is divided into a plurality of zones by means of a suitable division of the reaction volume, for example by dividing plates (cascaded stirred vessel) or combinations thereof.

The volume-specific power input per backmixed reaction zone should be not less than 0.1 W/I, preferably not less than 0.2 W/I and particularly preferably not less than 0.5 W/I. In general, up to 10 W/I are sufficient, preferably up to 5 W/I, particularly preferably up to 3 W/I and very particularly preferably up to 2 W/I The specific power input indicated is the power introduced per liter of reactor space volume of the reactor.

The power can be introduced via all possible types of stirrer, e.g. propeller, inclined blade, anchor, disk, turbine or beam stirrers. Preference is given to using disk and turbine stirrers.

In one embodiment, the reaction is carried out batchwise in a single stirred vessel. The abovementioned simultaneous addition or addition without a large time difference of the catalyst (C) is defined by the engineering and operating circumstances.

In a preferred embodiment, the reaction is carried out continuously. In this case, the components are preferably added simultaneously and continuously.

The reaction can optionally be carried out in at least one solvent and this can likewise be separated off together with the unreacted isocyanate (A) and, if appropriate, (B).

Preference is given to using no solvent.

To stop the reaction, use is made of a suitable deactivating agent in a molar ratio to the amount of catalyst (D) used of, for example, from 0.5 to 30, particularly preferably from 0.6 to 3, very particularly preferably from 0.8 to 2.

After the desired degree of conversion has been reached, the reaction is stopped by cooling the reaction mixture or preferably by thermal deactivation of the catalyst or by addition of a suitable deactivating agent.

The conversion can be chosen differently as a function of the isocyanate used. In general, a conversion (based on the reaction mixture before distillation) of from 10 to 60% (based on the NCO content before the reaction) is sought, preferably from 10 to 40%.

The deactivating agent is generally added at the reaction temperature but can also be added at a higher or lower temperature, for example up to 30° C. lower, preferably up to 20° C. lower and particularly preferably up to 10° C. lower.

Suitable deactivating agents are, for example, inorganic acids, e.g. hydrogen chloride, phosphorous acid or phosphoric acid, carboxylic acid halides, e.g. acetyl chloride or benzoyl chloride, sulfonic acids or esters, e.g. methanesulfonic acid, p-toluenesulfonic acid, methyl or ethyl p-toluenesulfonate, m-chloroperbenzoic acid and preferably dialkyl phosphates such as di-2-ethylhexyl phosphate and dibutyl phosphate. The use of deactivating agents comprising carbamate groups, as are described in the unpublished European patent application number 06125323.3 filed on Dec. 4, 2006, is also conceivable.

The addition is dependent on the type of deactivating agent. Thus, hydrogen chloride is preferably passed in gaseous form over or preferably through the reaction mixture; liquid deactivating agents are usually added in neat form or as a solution in a solvent which is inert under the reaction conditions and solid deactivating agents are added as such or as a solution or suspension in a solvent which is inert under the reaction conditions.

Thermal deactivation of the catalyst is generally only possible in the case of thermolabile catalysts (D), in particular in the case of catalysts which comprise an ammonium salt which bears a β-hydroxyalkyl-substituted radical. Such thermolabile catalysts frequently suffer an appreciable loss in activity at temperatures above 80° C., so that it is sufficient to heat the reaction mixture to temperatures above 90° C., preferably above 100° C. and particularly preferably above 120° C. In a preferred embodiment, this can be carried out during the removal of the unreacted isocyanate from the resulting reaction mixture by distillation:

The polyisocyanate-comprising reaction mixture prepared in this way is finally freed of any solvent or diluent present and preferably of excess, unreacted isocyanates in a manner known per se, for example by thin film distillation, at a temperature of from 90 to 220° C., if appropriate under reduced pressure, if appropriate while also passing an inert stripping gas through the reaction mixture, so that the polyisocyanates comprising isocyanurate groups are obtainable with a content of monomeric isocyanates of, for example, less than 1.0% by weight, preferably less than 0.5% by weight, particularly preferably less than 0.3% by weight, very particularly preferably less than 0.2% by weight and in particular not more than 0.1% by weight.

Apparatuses used for this purpose are flash, falling film, thin film and/or short path evaporators, which can, if appropriate, be superposed by a short column.

The distillation is generally carried out at a pressure in the range from 0.1 to 300 hPa, preferably below 200 hPa and particularly preferably below 100 hPa.

In a preferred embodiment, the distillation is carried out in a plurality of stages, for example from 2 to 5 stages.

The pressure is in this case advantageously reduced from stage to stage.

The temperature in the individual distillation stages is in each case in the range from 90 to 220° C.

The distillate of monomeric isocyanate which has been separated off is preferably recirculated to the reaction and reused, supplemented by freshly introduced isocyanate.

The freshly introduced isocyanate can advantageously be distilled or stripped with an inert, dry gas prior to the reaction.

If necessary, this recirculated distillate can be subjected to a treatment to improve the color number, for example a filtration via a filter, activated carbon or aluminum oxide.

The finished product can finally, if desired, be mixed with at least one solvent.

Examples of such solvents are aromatic and/or (cyclo)aliphatic hydrocarbons and mixtures thereof, and also preferably polar, aprotic solvents (and/or film formation aids).

As aromatic hydrocarbon mixtures, preference is given to those which comprise predominantly aromatic C₇-C₁₄-hydrocarbons and can comprise a boiling range from 110 to 300° C.; particular preference is given to toluene, o-, m- or p-xylene, trimethylbenzene isomers, tetramethylbenzene isomers, ethylbenzene, cumene, tetrahydronaphthalene and mixtures comprising such hydrocarbons. Such aromatic hydrocarbon mixtures are less preferred but can be present in minor amounts.

Examples of such hydrocarbon mixtures are the Solvesso® grades from ExxonMobil Chemical, in particular Solvesso® 100 (CAS No. 64742-95-6, predominantly C₉ and C₁₀ aromatics, boiling range about 154-178° C.), 150 (boiling range about 182-207° C.) and 200 (CAS No. 64742-94-5) and also the Shellsol® grades from Shell, Caromax® (e.g. Caromax® 18) from Petrochem Carless and Hydrosol from DHC (e.g. as Hydrosol® A 170). Hydrocarbon mixtures of paraffins, cycloparaffins and aromatics are also commercially available under the names Kristallöl (for example Kristallöl 30, boiling range about 158-198° C. or Kristallöl 60: CAS No. 64742-82-1), white spirit (for example likewise CAS No. 64742-82-1) or solventnaphtha (light: boiling range about 155-180° C., heavy: boiling range about 225-300° C.). The aromatics content of such hydrocarbon mixtures is generally greater than 90% by weight, preferably greater than 95% by weight, particularly preferably greater than 98% by weight and very particularly preferably greater than 99% by weight. It can be useful to use hydrocarbon mixtures having a particularly low content of naphthalene.

(Cyclo)aliphatic hydrocarbons are, for example, decalin, alkylated decalin and isomer mixtures of linear or branched alkanes and/or cycloalkanes, for example petroleum ether or ligroin, are less preferred but can be present in minor amounts.

The content of aliphatic hydrocarbons is generally less than 5% by weight, preferably less than 2.5% by weight and particularly preferably less than 1% by weight.

Preference is given to polar, aprotic solvents such as esters, ethers; glycol ethers and glycol esters, preferably of propylene glycol, particularly preferably of ethylene glycol, and also carbonates.

Esters are, for example, n-butyl acetate, ethyl acetate, 1-methoxypropyl 2-acetate and 2-methoxyethyl acetate, and also gamma-butyrolactone, 1,2-propylene carbonate, butyl glycol acetate, butyl diglycol acetate, dipropylene glycol dimethyl ether which is available as an isomer mixture under, for example, the trade name Proglyde® DMM from Dow Chemical Company.

Ethers are, for example THF, dioxane and also the dimethyl, diethyl or di-n-butyl ethers of ethylene glycol, diethylene glycol, triethylene glycol, propylene glycol, dipropylene glycol or tripropylene glycol.

Particular preference is given to n-butyl acetate, 1-methoxypropyl 2-acetate, 2-methoxyethyl acetate, N-methylpyrrolidone, gamma-butyrolactone, propylene carbonate (Solvenon® PC; 4-methyl-1,3-dioxolan-2-one), butoxyl (3-methoxy-n-butyl acetate), butyl glycol acetate, butyl diglycol acetate, dipropylene glycol dimethyl ether, propylene glycol diacetate, ethyl 3-ethoxypropionate and also dicarboxylic esters and mixtures thereof and, also mixtures of the solvents mentioned.

Very particular preference is given to n-butyl acetate, 1,2-propylene carbonate, butyl glycol acetate, butyl diglycol acetate, dipropylene glycol dimethyl ether and 3-methoxy-n-butyl acetate.

The choice of solvent makes it possible to influence, for example, the particle size, and the curing of the surface coating via the evaporation number. Butyl glycol acetate has, for example, a lower evaporation number than butyl diglycol acetate, so that the open time can be set via the ratio of the two and be reduced by means of the latter.

The water-emulsifiable polyisocyanates of the invention usually have a low viscosity which makes it easier for them to be incorporated in water and in coating compositions. In addition, the use of solvents can be reduced or even dispensed with as a result of the low-viscosity polyisocyanates, so that the coating compositions obtained using the polyisocyanates of the invention have a reduced content of volatile organic compounds (VOCs).

In general, the viscosity of the water-emulsifiable polyisocyanates obtained according to the invention is less than 4000 mPas, preferably less than 2000 mPas, particularly preferably less than 1500 mPas, very particularly preferably less than 1300 mPas and in particular in the range from 900 to 1200 mPas (in accordance with DIN EN ISO 3219 at 23° C. in a cone-and-plate rotational viscometer at a shear rate of 100 s⁻¹), so that dilution with solvents is not necessary.

The polyisocyanates obtained according to the invention have a favorable color number. Thus, the color number of the polyisocyanates obtained according to the invention, determined in accordance with DIN ISO 6271, is generally less than 100 APHA, preferably less than 80 APHA, particularly preferably less than 60 APHA, very particularly preferably less than 50 APHA and in particular less than 40 APHA.

The present invention further provides the production of coating compositions comprising the inventive water-emulsifiable polyisocyanates comprising isocyanurate groups by reaction with aqueous solutions, emulsions or dispersions of polyols: polyacrylatol, polyesterol, polyurethanol, polyetherol, polycarbonatol dispersions, and also their hybrids and/or mixtures of the polyols mentioned. The term hybrids refers to graft copolymers and other chemical reaction products which comprise chemically modified molecule parts having different (or identical) groups from among the groups mentioned.

Polyacrylatols can be prepared as primary or secondary dispersions, emulsions, solutions. These are prepared from olefinically unsaturated monomers. These are firstly comonomers having, for example, carboxyl, sulfonic acid and/or phosphonic acid groups as acid groups or their salts, e.g. (meth)acrylic acid, vinylsulfonic acid or vinylphosphonic acid. Secondly, they are hydroxyl-comprising comonomers such as hydroxyalkyl esters or amides of (meth)acrylic acid, e.g. 2-hydroxyethyl and 2- or 3-hydroxypropyl (meth)acrylate. Thirdly, they are unsaturated comonomers which have neither acid groups nor hydroxyl groups, e.g. alkyl esters of (meth)acrylic acid, styrene and derivatives, (meth)acrylonitrile, vinyl esters, vinyl halides, vinyl imidazole and others. The properties can be influenced, for example, via the compositon of the polymer or, for example, via the glass transition temperatures of the monomers (having different hardnesses).

Polyacrylatoles for aqueous applications are described, for example, in EP 358979 (U.S. Pat. No. 5,075,370), EP 557844 (U.S. Pat. No. 6,376,602), EP 1141066 (U.S. Pat. No. 6,528,573). An example of a commercially available secondary polyacrylate emulsion is Bayhydrol® A 145 (a product of Bayer MaterialScience). Examples of a primary polyacrylate emulsion are Bayhydrol® VP LS 2318 (a product of Bayer MaterialScience) and Luhydran® grades from BASF AG.

Other examples are Macrynal® VSM 6299w/42WA from Cytec, and Setalux® AQ grades from Nuplex Resins, e.g. Setalux® 6510 AQ-42, Setalux® 6511 AQ-47, Setalux® 6520 AQ-45, Setalux® 6801 AQ-24, Setalux® 6802 AQ-24, and Joncryl® from BASF Resins.

Polyesterols for aqueous applications are described, for example, in EP 537568 (U.S. Pat. No. 5,344,873), EP 610450 (U.S. Pat. No. 6,319,981) and EP 751197 (U.S. Pat. No. 5,741,849). Polyesterols for aqueous applications are, for example, WorléePol grades from Worlée-Chemie GmbH, Necowel® grades from Ashland-Südchemie-Kernfest GmbH, and Setalux® 6306 SS-60 from Nuplex Resins.

Polyurethanepolyol dispersions for aqueous applications are described, for example, in EP 469389 (U.S. Pat. No. 5,598,05). They are marketed, for example, under the trade name Daotan® from DSM NV.

Polyetherols for aqueous applications are described, for example, in EP 496210 (U.S. Pat. No. 5,304,400).

Hybrids and mixtures of the various polyols are described, for example, in EP 424705 (U.S. Pat. No. 4,179,98), EP 496205 (U.S. Pat. No. 5,387,642), EP 542085 (5,308,912), EP 542105 (U.S. Pat. No. 5,331,039), EP 543228 (U.S. Pat. No. 5,336,711), EP 578940 (U.S. Pat. No. 5,349,041), EP 758007 (U.S. Pat. No. 5,750,613), EP 751197 (U.S. Pat. No. 5,741,849), EP 1141065 (U.S. Pat. No. 6,590,028).

Polyesters/polyacrylates are described, for example, in EP 678536 (U.S. Pat. No. 5,654,391). An example of a secondary polyester/polyacrylate emulsion is Bayhydrol® VP LS 2139/2 (a product of Bayer MaterialScience).

Other polyols for aqueous applications are known under the following trade names: Macrynal®, Viacryl® from Cytec; Bayhydrol® from Bayer MaterialScience (polacrylate, polyurethane, polyester/polyacrylate, polyester/polyurethane, fatty-acid-modified polyester/polyacrylate, polycarbonate dispersions), Alberdingk® and Alberdur® from

Alberdingk Boley, Plusaqua® (alkyd, polyester, silicone polyesters) from Omya, Necowel® from Ashland-Südchemie-Kernfest GmbH (alkyd resins), Daotan® from DSM NV (formerly Solutia) (polyester, polycarbonate, fatty-acid-based, polyester-urethane-acrylic hybrids), Neocryl® (e.g. AF-10: acrylic-fluoro copolymer) from DSM NeoResins.

To incorporate the water-emulsifiable polyisocyanates according to the invention, it is generally sufficient to disperse the polyisocyanate obtained according to the invention in the aqueous dispersion of the polyol. An energy input of from 0 to not more than 10⁸ W/m³ is generally necessary to produce the emulsion.

The dispersions generally have a solids content of from 10 to 85% by weight, preferably from 20 to 70% by weight, and a viscosity of from 10 to 500 m Pas (measured at a temperature of 20° C. and a shear rate of 250 s⁻¹).

The mean particle size (z average) in the dispersion produced in this way, measured by means of dynamic light scattering using an Malvern® Autosizer 2 C, is generally<1000 nm, preferably<500 nm and particularly preferably<200 nm. The diameter is normally from 20 to 80 nm.

The polyisocyanates obtained according to the invention can be used for producing polyurethanes and polyurethane surface coatings, for example for one-component, two-component, radiation-curable or powder coating systems, and also the surface coating compositions produced therewith for coating various substrates, e.g. wood, wood veneer, paper, paperboard, card, film, textile, leather, nonwoven, plastic surfaces, glass, ceramic, mineral building materials and metals, each of which may optionally be precoated or pretreated.

In a use in coating compositions, the polyisocyanates according to the invention can be used, in particular, in primers, fillers, pigmented topcoats, undercoats and clear varnishes in the field of automobile repairs or in the surface coating of large vehicles. Such coating compositions are particularly useful for applications in which particularly high application reliability, exterior weathering resistance, optics and resistance to solvents, chemicals and water are required, e.g. in automobile repairs and the surface coating of large vehicles.

Such coating compositions are suitable as or in interior or exterior coatings, i.e.

applications in which they are exposed to daylight, preferably parts of buildings, decorative coatings, coatings on (large) vehicles and aircraft and industrial applications, bridges, buildings, electricity pylons, tanks, containers, pipelines, power stations, chemical plants, ships, cranes, posts, sheet piling, valves, pipes, fittings, flanges, couplings, halls, roofs, structural steel, furniture, windows, doors and parquet, can coating and coil coating. Such coating compositions are also suitable for floor coverings such as parking decks or in hospitals. In particular, the coating compositions according to the invention are used as or in clear varnishes, undercoats and topcoats for automobiles, primers and fillers, in particular in the refinish sector.

In a preferred embodiment, such coating compositions are used at temperatures ranging from ambient temperature to 80° C., preferably up to 60° C., particularly preferably up to 40° C. The articles coated are preferably ones which cannot be cured at temperatures which are too high, for example large machines, aircraft, large volume vehicles, wood and in refinish applications.

The isocyanate groups in the polyisocyanates according to the invention can optionally also be present in blocked form. Such groups for blocking are described in D. A. Wicks, Z. W. Wicks, Progress in Organic Coatings, 36, 148-172 (1999), 41, 1-83 (2001) and 43, 131-140 (2001).

Preferred groups for blocking are phenols, imidazoles, triazoles, pyrazoles, oximes, N-hydroxyimides, hydroxybenzoic esters, secondary amines, lactams, CH-acid cyclic ketones, malonic esters or alkyl acetoacetates.

These groups can be reacted in any way with the polyisocyanates according to the invention.

Imidazolic groups as groups which are reactive towards isocyanate groups, here referred to as “imidazoles” for short, are known, for example, from WO 97/12924 and EP 159117, triazoles are known from U.S. Pat. No. 4,482,721, CH-acid cyclic ketones are described, for example, in DE-A1 102 60 269, there in particular in paragraph [0008] and preferably in paragraphs [0033] to [0037], particularly preferably cyclopentanone-2-carboxylic esters and in particular ethyl cyclopentanone-2-carboxylate.

Preferred imidazoles are, for example, imidazoles which comprise a further functional group such as —OH, —SH, —NH—R, —NH₂, —CHO in addition to the free NH group, e.g. 4-(hydroxymethyl)imidazole, 2-mercaptoimidazole, 2-aminoimidazole, 1-(3-aminopropyl)-imidazole, 4,5-diphenyl-2-imidazolethiol, histamine, 2-imidazolecarboxaldehyde, 4-imidazolecarboxylic acid, 4,5-imidazoledicarboxylic acid, L-histidine, L-carnosine and 2,2′-bis(4,5-dimethylimidazole).

Suitable triazoles are 3-amino-1,2,4-triazole, 4-amino-1,2,4-triazole, 3,5-diamino-1,2,4-triazole, 1H-1,2,4-triazole-3-thiol, 5-methyl-1H-1,2,4-triazole-3-thiol and 3-amino-5-mercapto-1,2,4-triazole.

Preference is given to phenols, oximes, N-hydroxyimides, lactams, imidazoles, triazoles, malonic esters and alkyl acetonates, particularly preferably lactams, phenols, imidazoles, triazoles and malonic esters and very particularly preferably phenols.

It is an advantage of the polyisocyanates according to the invention that they have a low viscosity, improved dispersibility and/or an increased content of NCO groups compared to other water-emulsifiable polyisocyanates having a similar composition but having been prepared in another way.

While DE-A 3810908 does not disclose a trimerization of isocyanates in the presence of monofunctional polyalkylene oxides and merely describes the use of alcohols as cocatalysts in the trimerization of isocyanates, EP 56159 A1 discloses only the use of polyalkylene oxides as complexing agents for basic alkali metal compounds which can serve as catalyst for the trimerization of isocyanates.

Both documents do not recognize the advantages which the reaction according to the invention of isocyanate groups with alkoxylated monoalcohols (C) simultaneously with isocyanurate formation bring to the products formed. The disclosure content of DE-A 3810908 and EP 56159 Al gives not pointer to, for example, improved water-emulsifiability of the products obtained according to the invention.

The present invention therefore further provides for the use of alkoxylated monoalcohols (C) in the preparation of water-emulsifiable polyisocyanates from hexamethylene 1,6-diisocyanate (A) and optionally at least one further diisocyanate (B) with simultaneous formation of polyisocyanates comprising isocyanurate groups in the presence of at least one catalyst.

The water-emulsifiable polyisocyanates according to the invention can be used as crosslinkers for the production of surface coating compositions, adhesives and sealants. Thus, surface coating compositions, adhesives and sealants comprising at least one water-emulsifiable polyisocyanate according to the invention are likewise provided by the present invention.

### EXAMPLES

Percentages and ppm figures quoted in the present text are, unless indicated otherwise, % by weight and ppm by weight.

### General Method

Hexamethylene 1,6-diisocyanate (HDI) was placed in a flask provided with stirrer and reflux condenser and flushed with nitrogen for 2 hours. A monofunctional polyalkylene oxide as indicated in the table was subsequently added and the mixture was heated to 80° C. When the temperature had been reached, from 50 to 900 ppm of N-(2-hydroxypropyl)-N,N,N-trimethylammonium 2-ethylhexanoate (DABCO TMR®, from Air Products) was added.

No significant reaction between hydroxy groups and isocyanate groups or between the isocyanate groups takes place under these conditions. When the specified catalyst is used, the reaction proceeds significantly only above 80° C.

The reaction mixture was reacted at a temperature of 80° C. to the appropriate NCO value as shown in the table. The reaction mixture was stopped by means of a 1.5-fold excess of 95% strength bis(2-ethylhexyl) phosphate and the unreacted HDI was removed by distillation using a thin film evaporator.

The details of reaction time, NCO content of the product, monofunctional polyalkylene oxide and its amount are reported in the table.

| | | | | Alcohol | | | | | | |
|---|---|---|---|---|---|---|---|---|---|---|
| | | | Alcohol content | content of | | | | | | |
| | | | [g/100 g of | the | Amount of | | | | | |
| | | | HDI] before the | product | catalyst/ | Reaction | Conversion | Crude | | Viscosity |
| No. | Isocyanate | Alcohol | reaction | [% w/w] | HDI in ppm | time [min] | [%] | NCO [%] | NCO [%] | [mPa * s] |
| 1 | HDI | B | 4.48 | 11.72 | 100 | 20 | 36.6 | 38.5 | 20.0 | 1330 |
| 2 | HDI | B | 6.72 | 15.39 | 90 | 15 | 40.9 | 37.3 | 19.0 | 1230 |
| 3 | HDI | B | 6.67 | 14.80 | 100 | 10 | 42.2 | 37.0 | 20.1 | 792 |
| 4 | HDI + IPDI | B | 8.72 | 34.09 | 480 | 140 | 18.0 | 38.9 | 15.1 | 640 |
| | (3:1) | | | | | | | | | |
| 5 | IPDI | B | 6.67 | 14.90 | 800 | 10 | 41.8 | 28.0 | 14.5 | |

Crude NCO: NCO Content of the undistilled reaction mixture
NCO: NCO Content of the reaction mixture after removal of the unreacted isocyanate monomer

Polyether A is a methanol-initiated, monofunctional polyethylene oxide prepared using potassium hydroxide as catalyst and having an OH number of 112 measured in accordance with DIN 53 240, which corresponds to a molecular weight of 500 g/mol. The catalyst residues still present were subsequently neutralized with acetic acid. The basicity is determined by titration with HCl and is found to be 10.6 mmol/kg.

Polyether B is a methanol-initiated, monofunctional polyethylene oxide prepared using potassium hydroxide as catalyst and having an OH number of 112 measured in accordance with DIN 53 240, which corresponds to a molecular weight of 500 g/mol.

The catalyst residues still present were subsequently neutralized with acetic acid and salts were removed from the product. Here, potassium acetate formed is also removed.

### Comparative Example 1

Using a method analogous to Example 2 of WO 2005/047357, an isomer mixture of 80 parts of tolylene 2,4-diisocyanate and 20 parts of tolylene 2,6-diisocyanate were reacted completely, i.e. to an NCO content of 0% by weight, with polyether A. 1463 g of HDI are placed in a flask provided with stirrer and reflux condenser and flushed with nitrogen for 2 hours. 77 g of the urethane prepared and 100 ppm of DABCO TMR® are subsequently added, the mixture is heated to 80° C. and reacted to a crude NCO value of 37.0%. The reaction is stopped by addition of a 1.5-fold excess of 95% strength bis(2-ethylhexyl)phosphate to the reaction mixture and the unreacted HDI is removed by distillation using a thin film evaporator.

This gives a polyisocyanate which is not readily dispersible in water and has an NCO content of 19.5% and a viscosity of 2600 mPa*s.

### Comparative Example 2

2617 g of HDI were placed in a flask provided with stirrer and reflux condenser and flushed with nitrogen for 2 hours. 118 g of polyether B were subsequently added and the mixture was heated to 80° C. After 1 hour at 80° C., 100 ppm of DABCO TMR® were added and the reaction mixture was reacted at an external temperature of 85° C. to a crude NCO value of 38.5%.

The reaction was stopped by addition of a 1.5-fold excess of 95% strength bis(2-ethylhexyl)phosphate to the reaction mixture and the unreacted HDI was removed by distillation using a thin film evaporator.

This gave a polyisocyanate which displayed poor dispersibility in water compared to Example 1 and had an NCO content of 20.16% and a viscosity of 910 mPa*s.

### Comparative Example 3

1990 g of HDI were placed in a flask provided with stirrer and reflux condenser and flushed with nitrogen for 2 hours. 93 g of polyether B and 55 ppm of zinc acetylacetonate as allophanatization catalyst were subsequently added and the mixture was heated to 80° C. The slightly exothermic reaction was maintained at 80-85° C. by air cooling and the reaction was continued to a crude NCO value of 39.4%. The unreacted HDI was removed by distillation using a thin film evaporator.

This gave a water-dispersible polyisocyanate having an NCO content of 18.1% and a viscosity of 2800 mPa*s, which is significantly higher than in Example 2.

### Comparative Example 4 (Analogous to Example 1, EP 206 059)

1000 g of a commercial HDI isocyanurate (Basonat® HI 100 from BASF Aktiengesellschaft having an NCO value of 22.0% and a viscosity of 3000 mPa*s) were placed in a flask provided with stirrer and reflux condenser and flushed with nitrogen for about 30 minutes. 150 g of polyether B were subsequently added and the mixture was reacted at 110° C. for 2 hours. The reaction was stopped by addition of 0.31 g of paratoluenesulfonic acid.

This gave a water-dispersible polyisocyanate having an NCO content of 17.8% and a viscosity of 2300 mPa*s.

Compared to Example 3, the viscosity of this product is higher and the content of free NCO groups is lower.

### Comparative Example 5 (As Described in Example 5 of EP 524500)

2490 g of HDI were placed in a flask provided with stirrer and reflux condenser and flushed with nitrogen for 2 hours. 145 g of polyether B were subsequently added and the mixture was heated to 70° C. After 1 hour at 70° C., 263 ppm of benzyltrimethylammonium hydroid were added and the reaction mixture was reacted to a crude NCO value of 39.0%. The reaction was stopped by addition of a 1.5-fold excess of 95% strength bis(2-ethylhexyl)phosphate to the reaction mixture and the unreacted HDI was removed by distillation using a thin film evaporator.

This gave a water-dispersible polyisocyanate having an NCO content of 18.7% and a viscosity of 820 mPa*s.

Compared to Example 3, viscosity and content of free NCO groups of this product are comparable, but the product is more strongly colored.

Testing of the quality of incorporability:

4 g of the reaction product from the respective examples are weighed into a 50 ml penicillin bottle. The sample is then diluted to a solids content of 80% by means of 1 g of propylene carbonate. The resin solution is subsequently homogeneously colored by means of a drop of crystal violet (1% strength in isobutanol). 10 g of Macrynal® VSM 6299w/42WA (polyacrylateol emulsion having an OH number of 135 based on solid, product of Cytec) and 2.6 g of water are added in succession to this mixture. The mixture is subsequently stirred for 1.5 minutes by means of a 1 cm wide metal spatula and part of the mixture is cast onto a glass plate for assessment.

The assessment is carried out according to a scale of grades. Here:

Grade 1: good incorporability, uniform area

Grade 2: moderate incorporability, a few specks

Grade 3: poor incorporability, many specks

| | | Viscosity | |
|---|---|---|---|
| No. | NCO [%] | [mPa * s] | Dispersibility |
| Examples according | | | |
| to the invention | | | |
| 1 | 20.0 | 1330 | 1 |
| 2 | 19.0 | 1230 | 1 |
| 3 | 20.1 | 792 | 1 |
| 4 | 15.1 | 640 | 1 |
| Comparative examples | | | |
| 1 | 19.5 | 2600 | 3 |
| 2 | 20.1 | 910 | 2 |
| 3 | 18.1 | 2800 | 1 |
| 4 | 17.8 | 2296 | 3 |
| 5 | 18.7 | 820 | 1 |

### Use Examples

Two-component polyurethane coating compositions based on Macrynal® VSM 6299w (from Cytec Surface Specialties; index 150) and Bayhydrol® A 145 (from BayerMaterialScience) were produced from the polyisocyanates of Examples 1 to 5 and Comparative example 1. In the polyurethane coating compositions based on the polyisocyanates of Examples 1 to 5, the coatings became touch-dry more quickly. This touch-dryness was tested by means of a wad of absorbent cotton. The criterion for touch-dryness is whether the surface of the coating is no longer damaged on gentle contact.

1. A process for preparing water-emulsifiable polyisocyanates comprising isocyanurate groups, comprising: reacting
(A) a (cyclo)aliphatic diisocyanate,
(B) optionally at least one further diisocyanate, isocyanate and
(C) at least one alkoxylated monoalcohol
in the presence of at least one catalyst (D) which is able to accelerate the formation of isocyanurate groups from isocyanate groups, the reaction is stopped on reaching the desired conversion and the unreacted diisocyanate (A) and, if appropriate, diisocyanate (B) are separated off from the reaction mixture, wherein the amount of alkoxylated monoalcohol (C) after the end of the reaction and removal of unreacted (A) and, if appropriate, (B) is at least 1.0 mol % based on the ratio of hydroxy groups to the sum of all NCO groups from the components (A) and (B).

2. The process according to claim 1, wherein said (cyclo)aliphatic diisocyanate is selected from the group consisting of hexamethylene 1,6-diisocyanate (HDI), isophorone diisocyanate (IPDI), 4,4′-di(isocyanatocyclohexyl)methane, and 2,4′-di(isocyanatocyclohexyl)methane.

3. The process according to claim 1, wherein no diisocyanate (B) is present.

4. The process according to claim 1, wherein component (C) is a polyether alcohol represented by
R¹—O—[—Xᵢ—]ₖ—H
where
R¹ is C₁-C₂₀-alkyl, C₂-C₂₀-alkyl which may optionally be interrupted by one or more oxygen atoms, one or more sulfur atoms, one or more substituted or unsubstituted imino groups, or a combination thereof, C₆-C₁₂-aryl, C₅-C₁₂-cycloalkyl or a five- or six-membered heterocycle comprising at least one of oxygen, nitrogen and sulfur,
k is an integer from 5 to 40, and
each X₁ is independently selected from the group consisting of —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂—CHVin—O—, —CHVin—CH₂—O—, —CH₂—CHPh—O— and —CHPh—CH₂—O—,
where Ph is phenyl and Vin is vinyl.

5. The process according to claim 4, wherein R¹ is selected from the group consisting of methyl, ethyl, isopropyl, n-propyl, n-butyl, isobutyl, sec-butyl and tert-butyl.

6. The process according to claim 1, wherein catalysts comprising metal ions are excluded as catalyst (D).

7. The process according to claim 1, wherein the catalyst (D) is a quaternary ammonium salt represented by 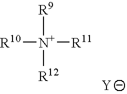
where
Y⊖=carboxylate (R¹³COO⁻), fluoride (F⁻), carbonate (R¹³O (CO)O⁻) or hydroxide (OH⁻),
where
R⁹ to R¹² are identical or different alkyl groups which have from 1 to 20 carbon atoms and may optionally be substituted by hydroxyl or phenyl groups and
R¹³ is hydrogen, a substituted or unsubstituted C₁-C₂₀-alkyl, a substituted or unsubstituted C₆-C₁₂-aryl or a substituted or unsubstituted C₇-C₂₀-arylalkyl.

8. The process according to claim 7, wherein
each R⁹ to R¹¹ radical is independently selected from the group consisting of methyl, ethyl and n-butyl, and
the R¹² radical is selected from the group consisting of methyl, ethyl, n-butyl, benzyl, 2-hydroxyethyl and 2-hydroxypropyl.

9. The process according to claim 1, wherein the catalyst (D) is deactivated by a deactivating agent selected from the group consisting of an inorganic acid, a carboxylic acid halide, a sulfonic acid, a sulfonic ester, m-chloroperbenzoic acid, a dialkyl phosphate, and a compound comprising at least one carbamate group.

10. The process according to claim 1, wherein the catalyst (D) is deactivated by heating to temperatures above 90° C.

11. A process for producing a polyurethane or a polyurethane surface coating operable for one-component, two-component, radiation-curable or powder coating systems, and in coating compositions for coating wood, wood veneer, paper, paperboard, card, film, textile, leather, nonwoven, plastic surfaces, glass, ceramic, mineral building materials and metals, each of which may optionally be precoated or pretreated, comprising reacting at least one water-emulsifiable polyisocyanate obtained by the process according to claim 1 with at least one polyol, wherein said at least one polyol is present in an aqueous solution, an emulsion, or a dispersion.

12. A process for producing a polyurethane or a polyurethane coating composition operable for coating interior or exterior coatings, parts of buildings, decorative coatings, bridges, buildings, electricity pylons, tanks, containers, pipelines, power stations, chemical plants, ships, cranes, posts, sheet piling, valves, pipes, fittings, flanges, couplings, halls, roofs and structural steel, windows, doors, parquet, can coating and coil coating, in particular for coatings on (large) vehicles and aircraft and industrial applications, floor coverings, parquet, clear varnishes, undercoats and topcoats for automobiles, primers and fillers, in particular in the refinish sector, comprising reacting at least one water-emulsifiable polyisocyanate obtained by the process according to claim 1 with at least one polyol, wherein said at least one polyol is present in an aqueous solution, an emulsion, or a dispersion.

13. A process for making a water-emulsifiable polyisocyanate, comprising reacting alkoxylated monoalcohols (C) in the with (cyclo)aliphatic diisocyanate (A) and optionally at least one further diisocyanate (B) with simultaneous formation of polyisocyanates comprising isocyanurate groups in the presence of at least one catalyst.

14. A surface coating composition, adhesive or sealant comprising at least one water-emulsifiable polyisocyanate obtained by a process according to claim 1.

15. The process according to claim 4, wherein k is an integer from 7 to 20.

16. The process according to claim 4, wherein k is an integer from 10 to 15.

17. The process according to claim 4, wherein each X₁ is independently selected from the group consisting of —CH₂—CH₂—O—, —CH₂—CH(CH₃)—O— and —CH(CH₃)—CH₂—O—.

18. The process according to claim 4, wherein each Xᵢ is —CH₂—CH₂—O—.
